(19) United States
(12) Patent Application Publication
(10) Pub. No.: US 2011/0041229 A1
(43) Pub. Date: Feb. 24, 2011

(54) HOT POX OUTDOOR GEAR

(76) Inventors: Michael Niemi, Wausau, WI (US); Scott Jenz, Wausau, WI (US)

Correspondence Address: LAW OFFICE OF JERRY D. HAYNES , P.A, P.O. Box 350392, Fort Lauderdale, FL, 33335, US

(21) Appl. No.: 12/542,972

(22) Filed: Aug. 18, 2009

(51) Int. Cl. A41D 1/00; A41D 27/20; A61F 7/00

(52) U.S. Cl. 2/69; 2/250; 607/108

(57) ABSTRACT

The present invention relates to apparel for use in a cold environment comprising: at least one pocket within the inner surface, where said at least one pocket includes a compartment for inserting a heating packet, where said compartment allows for adequate flow of oxygen to activate the heating packet. The compartment may include a mesh compartment. In one exemplary embodiment, the apparel for use in a cold environment may include: a plurality of pockets within the inner surface, where each pocket includes a mesh compartment, where said mesh compartment allows for adequate flow of oxygen to activate the heating packet; and at least one heating packet, where each heating packet is inserted in the mesh compartment of at least one of the pockets to provide thermal heat to the wearer. The apparel may include a glove, a mitten, a sweater, a jacket, a pair of pants, socks or shoes.

### BACKGROUND OF THE INVENTION

1. Field of Invention

The present invention relates to outdoor apparel that's designed to provide warmth and protection in a cold-weather environment.

2. Description of Related Art

Hunters, outdoorsmen, snowboarders, skiers, ice skaters and other winter outdoor activities expose individuals to cold and frigid weather. Furthermore some professions endure cold and frigid weather such as construction workers, postal workers, police officers, firemen or other occupations that require work outdoors. In order to keep warm many individuals must wear layered clothing and other winter apparel in order to remain warm while outside. One drawback to winter apparel is that the clothing may become thick, bulky and therefore impede the movement and mobility of the individual. Layered clothing takes a significant amount of time to put on and remove once inside.

Some apparel has been developed in order to address the needs of individuals who are outdoors during frigid weather. Some of the clothing includes the insertion of heat insulating chambers or pockets that may hold fluids or other materials that act as a thermal warming device. Most of the pocket-design apparel involves undergarments that are placed next to the skin and provide a means of activating the heating substances in the pockets of the apparel. One such garment is disclosed in U.S. Pat. No. 5,826,273 which discloses a thermal packet for effecting a heat transfer between the packet and the body of the user of the article of clothing that includes a torso enveloping portion conforming to the body of the user.

U.S. Pat. No. 5,054,127 discloses a system of pockets for an interchangeable system in which the pockets may be attached randomly about articles of clothing. The system disclosed in the '127 patent includes pockets that are removably attached and discusses a patch that may be permanently affixed to the clothing. The patches and pockets provide a means of heat transfer while being worn by an individual.

Further development of the articles of clothing that may provide warmth during cold weather may be needed to address some of the drawbacks of the prior art. Although the prior art provides some heating pads and pockets for coverage of the body, the pads and pockets fail to provide flexibility or adequate heat for long-term exposure to frigid and chilling weather.

### SUMMARY OF THE INVENTION

The present invention relates to apparel for use in a cold environment comprising: at least one pocket within the inner surface, where said at least one pocket includes a means for inserting a heating packet, where said means allows for adequate flow of oxygen to activate the heating packet. The means for inserting may include a mesh compartment. In one exemplary embodiment, the apparel for use in a cold environment may include: a plurality of pockets within the inner surface, where each pocket includes a mesh compartment, where said mesh compartment allows for adequate flow of oxygen to activate the heating packet; and at least one heating packet, where each heating packet is inserted in the mesh compartment of at least one of the pockets to provide thermal heat to the wearer. The apparel may include a glove, a mitten, a sweater, a jacket, a pair of pants, socks or shoes.

The present invention also may also provide maximum thermal heat, where each pocket may include a heating packet and where less than maximum thermal heat is desired, less than each pocket includes a heating packet.

### BRIEF DESCRIPTION OF DRAWINGS

FIG. 1A depicts the use of mesh heating pockets according to the present invention within gloves and mittens.

FIG. 1B shows apparel that includes of a plurality of mesh pockets according to the present invention.

FIG. 1C shows the use of mesh pockets on a cap.

FIG. 1D depicts an exemplary mesh pocket according to the present invention.

### DETAILED DESCRIPTION

The present invention provides a mesh pocket design (“Hot Pox”) that may be utilized on various apparel in order to hold warming packets that require adequate oxygen in order to activate the warming effect of the heating packet. The use of heating packets is known and it requires the flow of adequate oxygen in order to effectively use and activate the heating packet. The present invention provides a mesh pocket that may be placed over the inner surface of apparel for the insertion of heating packets and it effectively provides adequate oxygen to activate the warming effects of the heating packets.

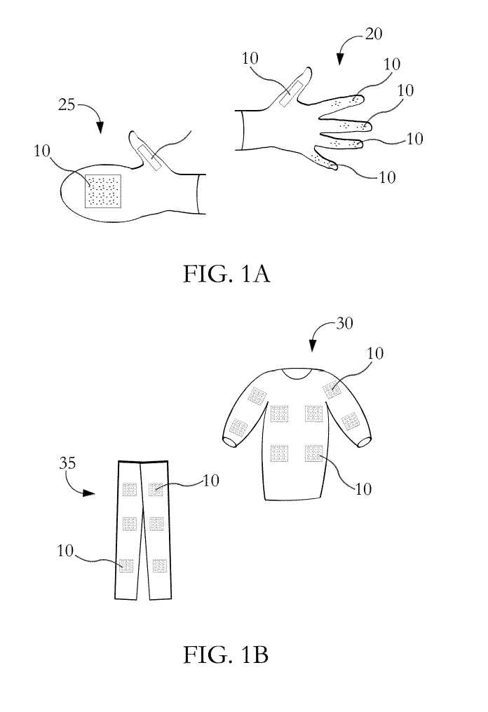

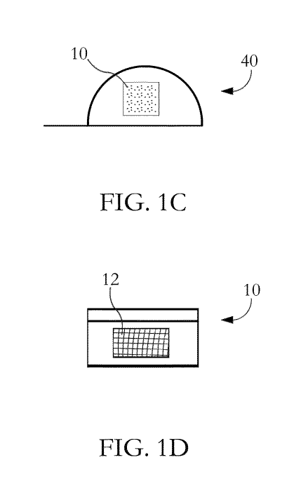

The mesh pocket (Hot Pox) according to the present invention may be placed on any particular article of clothing and provide a means for the insertion of a heating packet. FIG. 1D depicts a mesh Pocket 10 according to the present invention. The Pocket 10 includes a mesh Compartment 12 that provides a means for the insertion and removal of a heating packet. The use of warming packets can provide a very effective means for the supply of warmth and heat for up to eight hours if provided adequate oxygen flow to effectively use the chemicals within the heating packet. The mesh Compartment 12 therefore enables the adequate flow of oxygen onto the heating packet that may be inserted and therefore may be utilized on various outdoor apparel to provide warmth for the user.

FIG. 1A depicts the use of the mesh Pocket 10 according to the present invention on a Mitten 25 and a Glove 20. As depicted the mesh Pocket 10 may be placed within the palm or knuckle areas of the Mitten 25 or Glove 20. Miniature mesh Pockets 10 are displaced along the fingers of the Glove 20 and therefore each individual pocket may be designed to receive a miniature heating pack and therefore provide warmth for up to eight hours for a person's hands.

FIG. 1B depicts the use of the mesh Pocket 10 over some apparel specifically a pair of Pants 35 and a Sweater 30. A plurality of mesh Pockets 10 are provided over the surface of each the Sweater 30 and Pants 35. With such a widespread use of the mesh pockets a individual may place a number of heating packets over their entire body while using apparel as depicted in FIG. 1B. The mesh Pockets 10 according to the present invention receive a heating packet and the mesh design provides a means for the adequate flow of oxygen to the heating packet. A unique feature of the present invention is that although a plurality of mesh pockets are provided over the inner surface of both the Sweater 30 and Pants 35, a user may choose not use each pocket and therefore control the amount of heat that is supplied by either using a greater number or a lesser number of heating packs in the available mesh pockets.

FIG. 1C depicts the use of a mesh Pocket 10 on a Cap 40. As depicted this Cap 40 may provide heat to an individual's head area while in a frigid environment. As provided and depicted in the foregoing figures a individual may utilize a number of heating packs essentially from head to toe through the use of apparel that includes the mesh Pocket 10 according to the present invention. It is further seen that the individual can control the number of pockets that are utilized and therefore control the amount of heat provided while in a cold environment.

The mesh pocket design (“Hot Pox”) according to the present invention may be utilized in order to protect the body from frigid weather conditions and also provides the user with a unique discretionary control of the pockets. Consequently for highly frigid or low temperatures an individual may place the heating packets in each pocket of their apparel and therefore provide adequate heat for up to eight hours. In even less frigid or cold environments an individual may not use the entire set of mesh pockets and therefore vary the amount of heat that's necessary to endure the weather conditions. The mesh pocket apparel according to the present invention provides pockets that can be utilized on clothing such as jackets, sweaters, pants, hats, gloves and even footwear such as boots or socks. The apparel according to the present invention is particularly useful for outdoor enthusiasts who participate in activities such as skiing, hunting, fishing or for occupations that must endure cold weather environments.

What is claimed is:

1. Apparel for use in a cold environment comprising:
a. at least one pocket within the inner surface, where said at least one pocket includes a means for inserting a heating packet, where said means allows for adequate flow of oxygen to activate the heating packet.

2. The apparel according to claim 1, where said means for inserting includes a mesh compartment.

3. Apparel for use in a cold environment comprising:
a. a plurality of pockets within the inner surface, where each pocket includes a mesh compartment, where said mesh compartment allows for adequate flow of oxygen to activate the heating packet; and
b. at least one heating packet, where each heating packet is inserted in the mesh compartment of at least one of the pockets to provide thermal heat to the wearer.

4. The apparel according to claim 3, where the apparel includes at least one of a glove, a mitten, a sweater, a jacket, a pair of pants, socks and shoes.

5. The apparel according to claim 3, where each pocket includes a heating packet such that the apparel provides maximum thermal heat.

6. The apparel according to claim 3, where less than each pocket includes a heating packet such that the apparel provides less than maximum thermal heat.

7. The apparel according to claim 4, where the glove includes miniature pockets in each finger opening of the glove.

8. The apparel according to claim 3, where said apparel provides thermal heat up to an eight-hour period.
